(19) United States
(12) Patent Application Publication
(10) Pub. No.: US 2011/0264176 A1
(43) Pub. Date: Oct. 27, 2011

(54) HOT TIP VEIN THERAPY DEVICE

(76) Inventors: Jerome Jackson, Los Altos, CA (US); Steven H. Trebotich, Newark, CA (US); Grant Michael Glaze, Sunnyvale, CA (US); Chun-Chih Cheng, Sunnyvale, CA (US); Joseph Michael Tartaglia, Morgan Hill, CA (US); Fiona M. Sander, Los Altos Hills, CA (US)

(21) Appl. No.: 13/126,513

(22) PCT Filed: Nov. 18, 2009
(86) PCT No.: PCT/US09/64894

§ 371 Date: Jul. 18, 2011

Related U.S. Application Data
(60) Provisional application No. 61/115,864, filed on Nov. 18, 2008.
(60) Provisional application No. 61/228,298, filed on Jul. 24, 2009.

(51) Int. Cl. A61F 7/12

(52) U.S. Cl. 607/105

(57) ABSTRACT

Methods and apparatus for generating vapor within a catheter are provided which may include any number of features. One feature is generating vapor with an electrode array within a catheter. Another feature is sensing an impedance of the electrode array, and adjusting the power delivered to the electrode array to fully generate vapor within the catheter. Another feature is delivering the vapor to a vein of a patient for vein reduction therapy.

### CROSS REFERENCE TO RELATED APPLICATIONS

This application claims the benefit under 35 U.S.C. 119 of U.S. Provisional Patent Application No. 61/115,864, filed Nov. 18, 2008, titled “Hot Tip Vein Therapy Device,” and U.S. Provisional Patent Application No. 61/228,298, filed Jul. 24, 2009, titled “Hot Tip Vein Therapy Device.” These applications are herein incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

The human venous system of the lower limb consists essentially of the superficial venous system and the deep venous system with perforating veins connecting the two systems. The superficial system includes the great saphenous, small saphenous and the lateral saphenous systems. The deep venous system includes the anterior and posterior tibial veins which unite to form the popliteal vein, which in turn becomes the femoral vein when joined by the short saphenous vein.

The venous systems contain numerous one-way valves for facilitating blood flow back to the heart. Venous valves are usually bicuspid valves, with each cusp forming a sack or reservoir for blood which, under pressure, forces the free surfaces of the cusps together to prevent retrograde flow of the blood and allow antegrade flow to the heart. When an incompetent valve is in the flow path of retrograde flow toward the foot, the valve is unable to close because the cusps do not form a proper seal and retrograde flow of blood cannot be stopped.

Incompetence in the venous system can result from vein dilation, which causes the veins to swell with additional blood. Separation of the cusps of the venous valve at the commissure may occur as a result. The leaflets are stretched by the dilation of the vein and concomitant increase in the vein diameter which the leaflets traverse. Stretching of the leaflets of the venous valve results in redundancy which allows the leaflets to fold on themselves and leave the valve open. This is called prolapse, which can allow reflux of blood in the vein. Eventually the venous valve fails, thereby increasing the strain and pressure on the lower venous sections and overlying tissues. Two venous diseases which often involve vein dilation are varicose veins and chronic venous insufficiency.

The varicose vein condition includes dilatation and tortuosity of the superficial veins of the lower limb, resulting in unsightly protrusions or discoloration, ‘heaviness’ in the lower limbs, itching, pain, and ulceration. Varicose veins often involve incompetence of one or more venous valves, which allow reflux of blood from the deep venous system to the superficial venous system or reflux within the superficial system.

Current varicose vein treatments include invasive open surgical procedures such as vein stripping and occasionally vein grafting, venous valvuloplasty and the implantation of various prosthetic devices. The removal of varicose veins from the body can be a tedious, time-consuming procedure and can be a painful and slow healing process. Complications including scarring and the loss of the vein for future potential cardiac and other by-pass procedures may also result. Along with the complications and risks of invasive open surgery, varicose veins may persist or recur, particularly when the valvular problem is not corrected. Due to the long, arduous, and tedious nature of the surgical procedure, treating multiple venous sections can exceed the physical stamina of the physician, and thus render complete treatment of the varicose vein conditions impractical.

Newer, less invasive therapies to treat varicose veins include intralumenal treatments to shrink and/or create an injury to the vein wall thereby facilitating the collapse of the inner lumen. These therapies include sclerotherapy, as well as catheter, energy-based treatments such as laser, Radio Frequency (RF), or resistive heat (heater coil) that effectively elevate the temperature of the vein wall to cause collagen contraction, an inflammatory response and endothelial damage. Sclerotherapy, or delivery of a sclerosant directly to the vein wall, is typically not used with the larger trunk veins due to treatment complications of large migrating sclerosant boluses. Laser energy delivery can result in extremely high tissue temperatures which can lead to pain, bruising and thrombophlebitis. RF therapy is typically associated with lengthy treatment times, and resistive heater coil treatments can be ineffective due to inconsistent vein wall contact (especially in larger vessels). The catheter based treatments such as laser, resistive heater coil and RF energy delivery also typically require external vein compression to improve energy coupling to the vein wall. This is time consuming and can again lead to inconsistent results. In addition, due to the size and/or stiffness of the catheter shaft and laser fibers, none of these therapies are currently being used to treat tortuous surface varicosities or larger spider veins. They are currently limited in their use to large trunk veins such as the great saphenous vein (GSV). Tortuous surface varicosities are currently treated with sclerotherapy and ambulatory phlembectomy, while larger spider veins are currently only treated with sclerotherapy.

U.S. Patent Appl. Publ. No. 2002/0177846 describes a catheter-based vapor system for use, e.g., in treating varicose veins. In one embodiment, described in connection with FIG. 19 of that publication, the device generates vapor in a larger diameter chamber proximal to a smaller diameter catheter and catheter outlet. The disclosure of this patent publication is incorporated herein by reference.

U.S. Pat. No. 6,911,028 also describes a catheter-based vapor system for use in shrinking or otherwise modifying veins and other lumens. In one embodiment, electrodes of opposite polarity in a recessed bore near the distal end of the catheter generate and eject pressurized vapor into the vein or other lumen. The disclosure of this patent is incorporated herein by reference.

### SUMMARY OF THE INVENTION

In one embodiment, a method for generating steam within a catheter comprises delivering a fluid to a vapor generation chamber, delivering power to an electrode array disposed on or in the vapor generation chamber, sensing a signal of the electrode array, and adjusting the power delivered to the electrode array based on the sensed signal to fully generate vapor in the vapor generation chamber.

In one embodiment, the sensed signal is an impedance. The power delivered to the electrodes can be decreased as the sensed impedance increases.

In another embodiment, the sensed signal is a fluid flow rate. In yet another embodiment, the sensed signal is a steam temperature.

In some embodiments, the electrode array is a bipolar electrode array.

In one embodiment, the fluid is delivered at a constant flow rate. In another embodiment, the fluid is delivered at a time varying rate. In yet another embodiment, the fluid is delivered at a rate dependent upon the diameter of the vessel or organ being treated.

The method can further comprise delivering vapor to a patient. The vapor can be delivered to a vein of a patient. In some embodiments, delivering the vapor to the vein reduces a lumen of the vein.

In some embodiments, the power delivered to the electrode array is adjusted automatically by a controller. In other embodiments, the power is adjusted manually.

In one embodiment, the fluid is saline. In other embodiments, the fluid is electrically conductive.

Another method of delivering therapy to a vein is provided, comprising inserting a catheter into the vein, delivering a fluid to a vapor generation chamber in the catheter, delivering power to an electrode array disposed on the vapor generation chamber, sensing a signal of the electrode array, adjusting the power delivered to the electrode array based on the sensed impedance to fully generate vapor in the vapor generation chamber, and delivering the fully generated vapor to the vein.

In one embodiment, the sensed signal is an impedance. The power delivered to the electrodes can be decreased as the sensed impedance increases.

In another embodiment, the sensed signal is a fluid flow rate. In yet another embodiment, the sensed signal is a steam temperature.

In some embodiments, the electrode array is a bipolar electrode array.

In one embodiment, the fluid is delivered at a constant flow rate. In another embodiment, the fluid is delivered at a time varying rate. In yet another embodiment, the fluid is delivered at a rate dependent upon the diameter of the vessel or organ being treated.

The method can further comprise delivering vapor to a patient. The vapor can be delivered to a vein of a patient. In some embodiments, delivering the vapor to the vein reduces a lumen of the vein.

In some embodiments, the power delivered to the electrode array is adjusted automatically by a controller. In other embodiments, the power is adjusted manually.

In one embodiment, the fluid is saline. In other embodiments, the fluid is electrically conductive.

A vapor generating device is provided, comprising a vapor generation chamber, the vapor generation chamber having an electrode array disposed therein, a fluid reservoir coupled to the vapor generation chamber, an RF generator coupled to the electrode array, a controller configured to sense an impedance of the electrode array, wherein the vapor generation chamber is adapted to transform a fluid from the fluid reservoir into a fully developed vapor within the device.

In some embodiments, the electrode array comprises a flattened configuration. In another embodiment, the electrode array is disposed along an inner circumference of the catheter.

The vapor generation device can further comprise a second vapor generation chamber.

In some embodiments, the fluid is saline.

In one embodiment, the controller automatically adjusts a power delivered by the RF generator to the electrode array based on the sensed impedance.

In one embodiment, the vapor generation device further comprises a delivery needle coupled to the vapor generation chamber.

### BRIEF DESCRIPTION OF THE DRAWINGS

FIG. 1 is a schematic view of a vapor therapy system.

FIG. 2 is close up view of a catheter for use in the vapor therapy system of FIG. 1.

FIGS. 3a-3b illustrate an electrode array disposed in a catheter of a vapor therapy system.

FIGS. 4a-4b illustrate another embodiment of an electrode array disposed in a catheter of a vapor therapy system.

FIG. 5 illustrates another embodiment of a vapor therapy system.

FIGS. 6a-6c illustrate a vapor generation chamber adapted to generate a high temperature vapor or steam.

FIG. 7 illustrates another embodiment of an electrode array disposed in a catheter of a vapor therapy system.

FIGS. 8a-8b illustrate another embodiment of an electrode array disposed in a catheter of a vapor therapy system.

### DETAILED DESCRIPTION OF THE INVENTION

One embodiment of the invention provides a catheter-based vapor therapy system that generates vapor at a distal end. The distal tip of the catheter is small enough to fit within not only the GSV but also in smaller vessels within the patient's legs. The vapor therapy system can be used to treat varicose veins or for vein lumen reduction therapy. Vapor generation catheters according to this embodiment may be made with diameters in the range of 4 Fr to 10 Fr., which is useful for treating a common range of veins and/or blood vessels, including the GSV and major tributaries emanating from it, since the catheter's diameter will easily fit within these vessels. However, in other embodiments, the catheters can be larger than 10 Fr or smaller than 4 Fr.

One aspect of vapor therapy systems according to this embodiment is the heat source at the distal end of the catheter that creates the liquid to vapor phase change, particularly the dimensions and efficiency of the heat source. In order to place the tip of the vapor catheter in a small lumen, such as a leg vein smaller than the GSV, a catheter diameter of 7 Fr or less may be desirable. While it might be possible to generate vapor outside the patient and deliver it via a catheter to the treatment site, generation and delivery remote from the treatment site raises issues with respect to protection of both patient and clinician from bums and with respect to the quality or latent energy of the vapor delivered. Generation of vapor at the catheter's distal outlet, however, raises issues with respect to the size and configuration of the heat source. In particular, the heat source must not only be small enough to fit within the catheter tip, it must also permit sufficient liquid and vapor flow to provide the desired therapeutic benefit.

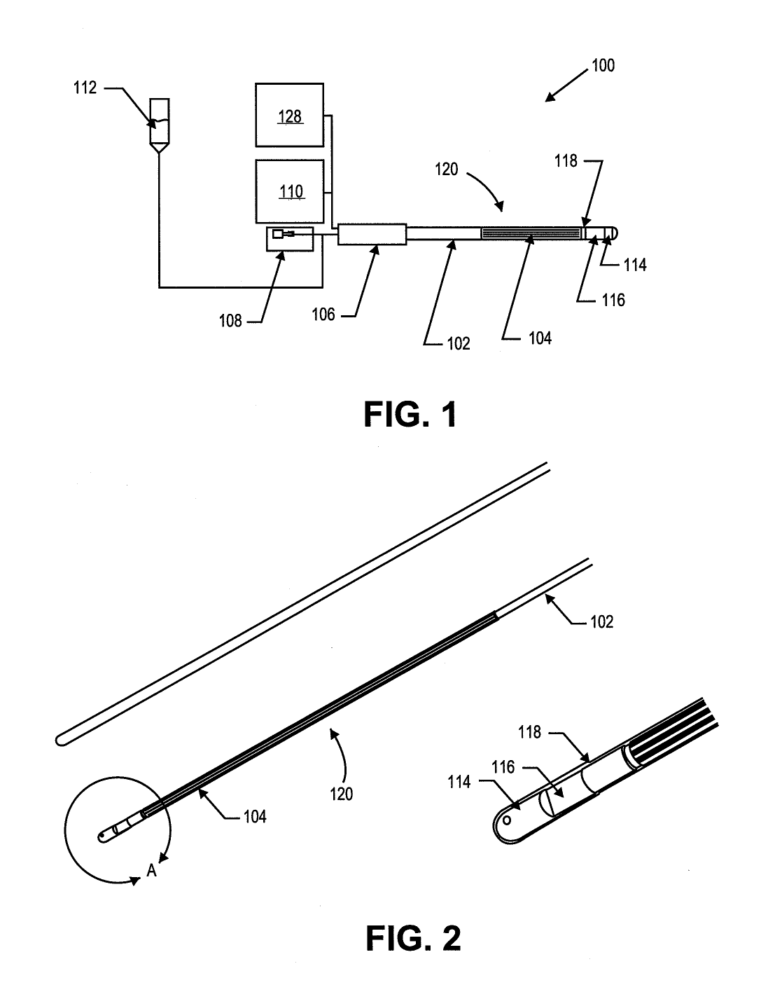

FIGS. 1 and 2 illustrate a vapor therapy system 100 according to one embodiment of the invention. System 100 includes catheter 102, electrode array 104, handpiece 106, pump 108, RF generator 110, fluid reservoir 112, and vapor port(s) 114. System 100 may further include flow restrictor 116 and sensors 118.

In this embodiment, fluid can be provided from a fluid reservoir 112 to catheter 102 by pump 108, which can be a positive displacement pump, a peristaltic pump, a syringe pump, or other fluid metering system, for example. The fluid may be, e.g., normal sterile (0.9%) saline. Other suitable fluids are those that are electrically conductive and can include other concentrations of sterile saline, such as hypertonic concentrations of 1%, 2%, etc., and hypotonic concentrations of 0.8%, 0.7%, etc., fluids containing other ionic molecules, such as potassium chloride, etc. In addition to a pump, the fluid can be dispensed from a pressurized reservoir, such as a saline bag with a pressure cuff, and be metered out precisely with a valve. The pump or other pressurized fluid source can deliver fluid to the catheter, where it enters a fluid delivery lumen that extends from a liquid inlet to a vapor generation chamber 120 within the catheter. Prior to use to shrink leg veins to treat varicose veins, the saline or other liquid can be provided from the fluid reservoir (such as a saline drip bag) to the pump and injected into the catheter at low flow rates of, for example, 1 drop to 15 drops per minute in a stand-by mode to prevent retrograde flow of blood or other physiologic material into the catheter and to prevent clotting within or near the catheter. Alternatively, an anticoagulant, such as heparin, can be added to the fluid drip which enhances its ability to prevent clots in or near the catheter body. Liquid can drip out of the vapor ports of the vapor generation catheter in the stand-by mode. In an alternative configuration, the electrode can be a microwave emitter, an inductive heater, a photonic device, such as a laser diode, light emitting diode, or other device that delivers photonic (light) energy and the fluid used to generate the vapor would not need to be conductive.

The catheter can be adapted to be inserted into the vessels of a patient's legs. In one embodiment, the outer diameter of the catheter can be formed from polyimide or from any other suitable material, such as Pebax, Polyamide, nylon, Hytrel, or other biocompatible plastic, rubber, thermoset, thermoplastic, or elastomeric material. The catheter shaft can also be braided to increase kink resistance and column strength or pushability.

The distal tip of the catheter has one or more vapor exit ports 114 downstream of or distal to the flow restrictor. For example, the vapor generation catheter shown in FIGS. 1 and 2 can have a stainless steel tip with side vapor exit ports formed therein. Other vapor exit ports may be arranged around the circumference and/or on the distal end, as needed. In addition, the vapor port can be on the leading end of the catheter to allow the vapor s\to be dispensed forward. Some suitable vapor exit port configurations as shown, for example, in U.S. Ser. No. 61/059,518, the disclosure of which is incorporated herein by reference.

In the embodiment illustrated in FIG. 1, an optional handpiece 106 can be provided at the proximal end of the catheter for convenience in placing and moving the catheter. Connections to the liquid source and to the power source may be made through the handpiece. The length of the handpiece and of the catheter itself can be modified to fit the intended application. For example, to treat veins within human legs, the length of the catheter distal to the handpiece may be between 10 cm and 100 cm, however longer lengths to accommodate longer legs can be fabricated.

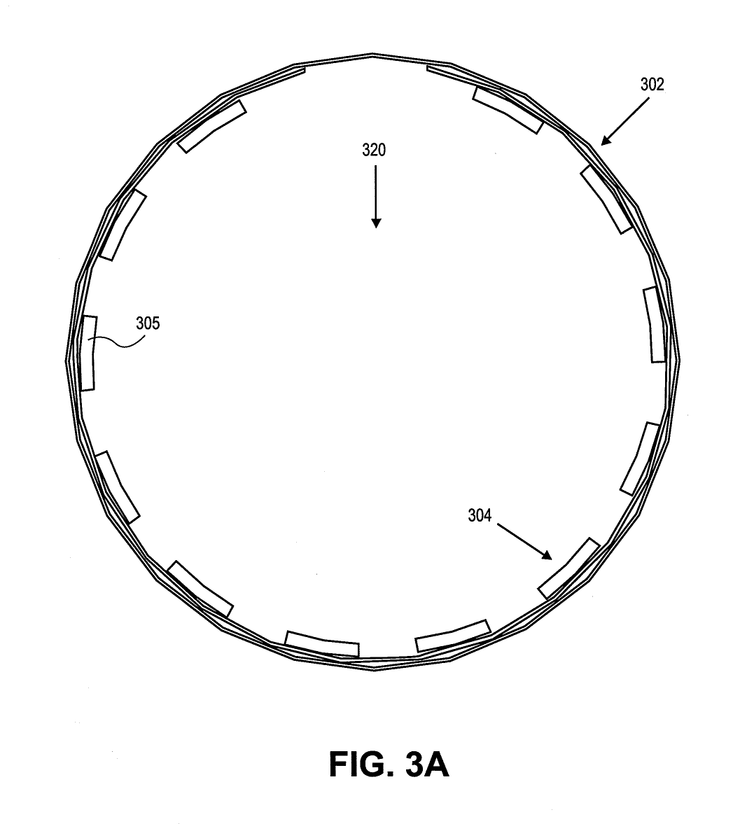

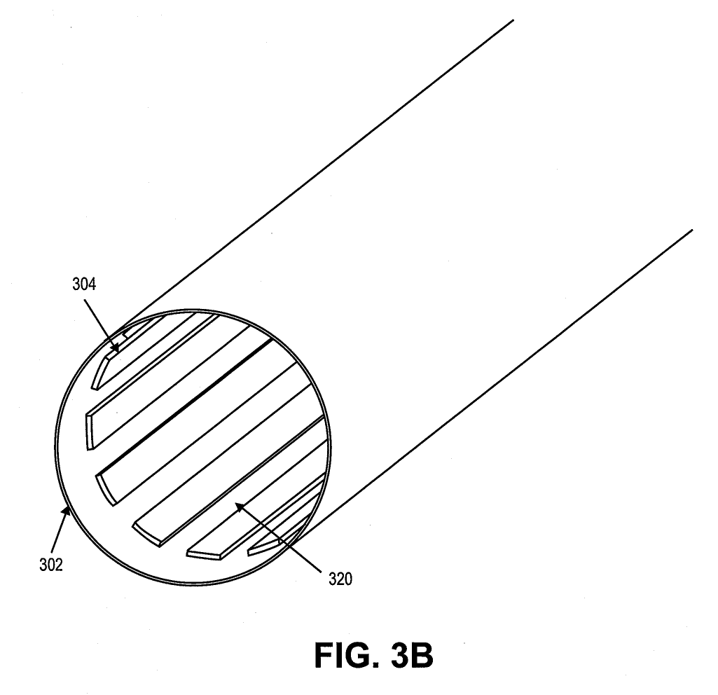

As shown in FIGS. 1-2, the vapor generation chamber 120 can include an electrode array 104 having a particularly small form factor. The vapor generation chamber can be disposed in a distal portion of the catheter 102 and be in fluid communication with the fluid reservoir 512 and vapor ports 514. The vapor generation chamber can also be located anywhere within the length of the catheter shaft, or even in the handpiece. The electrode array can be a bipolar electrode or bipolar electrode array, a monopolar electrode or monopolar electrode array, or a combination of a bipolar and monopolar electrode array, for example. In some embodiments, the electrode array comprises a plurality of bipolar electrodes in a flattened configuration. The electrode pairs can be formed as conductive traces within the catheter. In one embodiment, the electrode pairs can be formed as 0.010 to 0.015 inch wide strips separated by a 0.010 to 0.015 inch wide spacing by 6 inches long 2 oz. copper traces on a thin polyimide film, for example. As shown in FIGS. 3a and 3b, the electrode array can comprise 6 electrode pairs. However, in other embodiments, any number of electrode pairs can be used.

FIGS. 3a-3b illustrate one embodiment of an electrode array 304 disposed in a distal portion of catheter 302. The electrode array comprises a plurality of electrodes 305 positioned along an inner circumference of the catheter to form a vapor generation chamber 320. As shown in FIG. 3a, the electrode array 304 can comprise 6 pairs of flattened electrodes. In other embodiments the electrode array can include any number of electrodes 305. The electrode array as shown in FIGS. 3a-3b may extend 15 cm along the length of catheter 302, for example. The electrode array can be configured to generate a high temperature vapor or steam in vapor generation chamber 320.

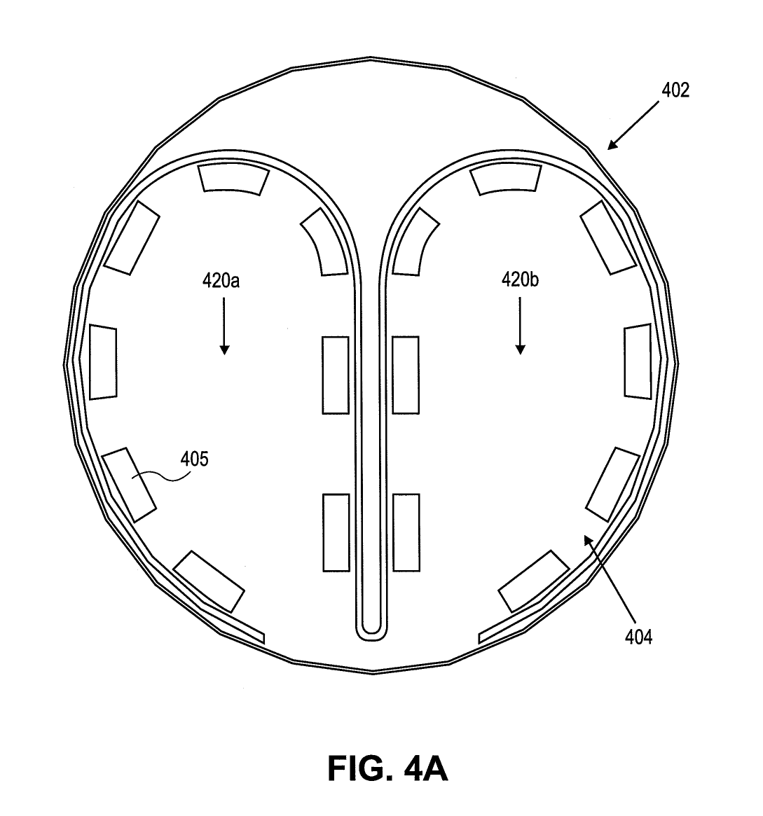

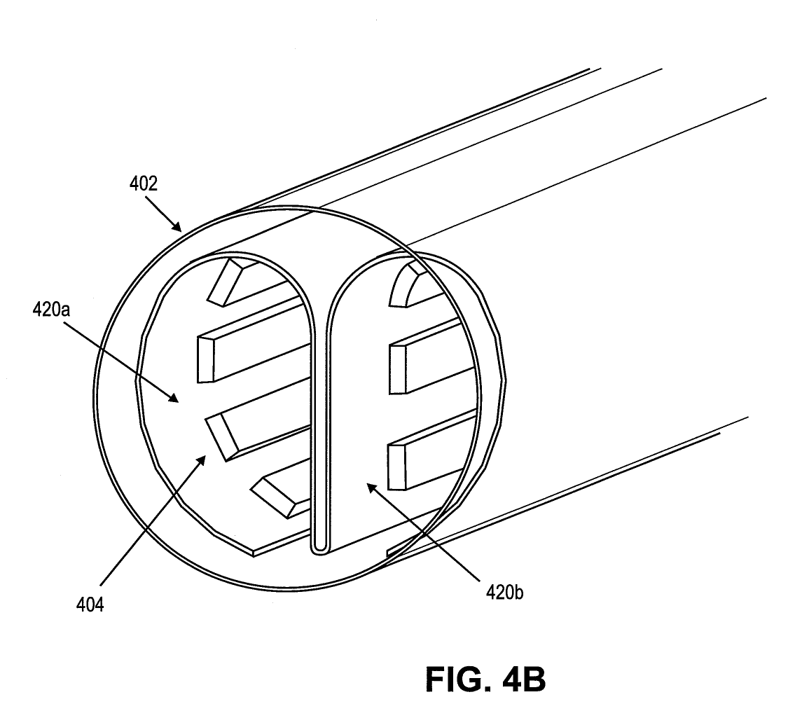

FIGS. 4a-4b illustrate an alternative embodiment of an electrode array 404 disposed in a distal portion of catheter 402. The electrode array comprises a plurality of electrodes 405 positioned to form a pair of vapor generation chambers 420a and 420b. As shown in FIG. 4a, the electrode array 404 can comprise 8 pairs of electrodes. However, in other embodiments the electrode array can include any number of electrodes 405. The electrode array can be configured to generate a high temperature vapor or steam in vapor generation chambers 420a and 420b.

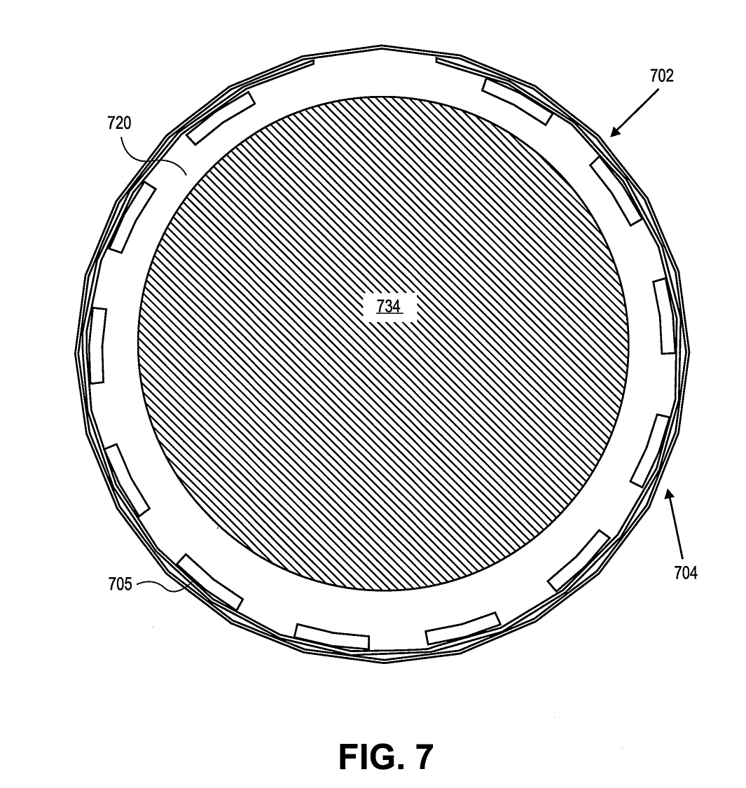

FIG. 7 illustrates another embodiment of an electrode array 704 disposed in a distal portion of catheter 702. The electrode array comprises a plurality of electrodes 705 positioned to form a vapor generation chamber 720, as described above. Additionally, a non-conductive filler material 734 can be placed axially within the vapor generation chamber to increase the energy density. The filler material 734 can be flexible, such as a silicone rod or polyimide tube with plugged ends, for example. Since the energy density is highest in the regions near the electrodes, and lowest near the center axis of the vapor generation chamber, the filler material can displace fluid in the low energy density regions (i.e., near the center of the vapor generation chamber) and force the fluid to flow only in high density regions (i.e., near the electrodes). Additionally, the non-conductive filler will not short out any electrode that it may come into contact with.

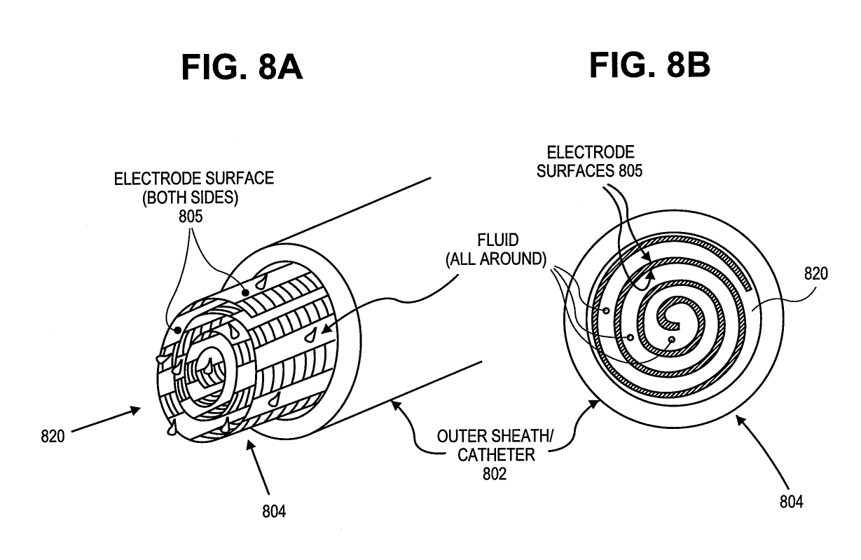

FIGS. 8a-8b illustrate an alternative embodiment of an electrode array 804 disposed in a distal portion of catheter 802. The electrode array comprises a plurality of electrodes 805 positioned to form a vapor generation chamber 820. As shown in FIGS. 8a-8b, the electrode array 804 can comprise a coiled sheet housing electrodes 805 on both the top and bottom surfaces. In another embodiment, the electrodes can be on only one side of the coiled sheet (i.e., on either the top or bottom of the sheet). The sheet can be spiral wound and placed within the catheter 802. Conductive fluid, as described above, can be delivered to the vapor generation region 820 between the coiled sheet of electrodes. This particular embodiment can maximize the electrode surface within catheter 802 thereby maximizing the heat generation capability of the catheter. Since more electrodes come into contact with fluid in this embodiment, more fluid can be heated in a given amount of time, which can maximize vapor delivery. Referring back to FIG. 1, the electrode arrays described herein can be connected to an energy source, such as RF generator 110. In one embodiment, the RF generator can be a 350 W, 460 kHz, RF generator. In another embodiment, the energy source can be a microwave generator operating at, for example 915 MHz or 2.45 GHz. In another embodiment, the energy source can be a power supply to provide energy to the photonic device. Other suitable RF, microwave or photonic sources may be used, as appropriate. In some embodiments, the system can include an optional controller 128. The controller can be integral to the RF generator, or can be a separate system component. In some embodiments, the system can include sensors, such as temperature or pressure sensors (not shown). The system, such as the controller or the sensors, can monitor the flow rate of fluid delivered to the catheter, the power levels and frequency of the generator, the impedance and/or resistance of the electrode array, and the temperature of the electrode array or the fluid within the vapor generation chamber. In some embodiments, the controller can adjust the parameters of the system, such as the fluid flow rate or the power of the RF generator, based on a sensed signal, such as a sensed impedance, resistance, conductance, flow rate, temperature, pressure, refractive index, mass or volume flow. In other embodiments, the parameters can be adjusted manually.

The electrode array can surround a vapor generation chamber 120 disposed at the outlet of the liquid supply lumen. As shown in FIGS. 3a-3b and 4a-4b, the vapor generation chamber can be positioned at a distal portion of the catheter. A flow restrictor 116 can be disposed between the vapor generation chamber and a vapor port 114. The flow restrictor can be sized to ensure that the pressure within the vapor generation chamber is high enough so that the vapor is superheated when it exits the catheter. The vapor can be in the range of 100 to 140 degrees when it exits the catheter, or higher if higher steam quality is desired. In the embodiment shown in FIGS. 1 and 2, the flow restrictor is formed from porous PTFE bonded in place within the catheter using high-temperature adhesive. In other embodiments, a metal based filter material, duckbill valve, ball checkvalve, micro-lumens or a flow control orifice may be used as the flow restrictor.

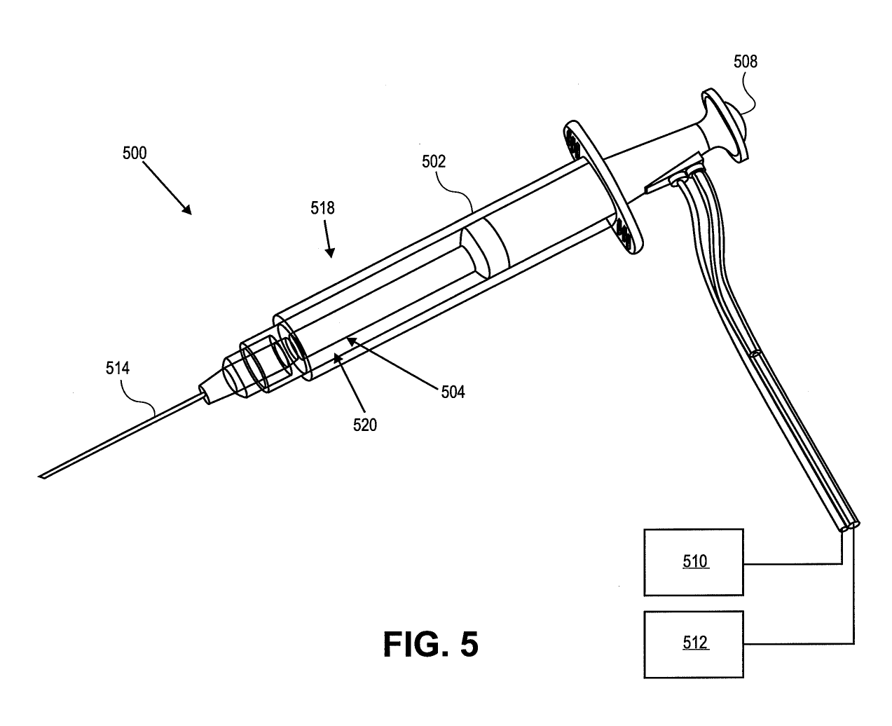

FIG. 5 illustrates a vapor therapy system 500 according to one embodiment of the invention. System 500 includes body 502, electrode array 504, plunger (pump) 508, RF generator 510, fluid reservoir 512, and delivery needle 514. System 500 may further include sensors 518. Electrode array 504, RF generator 510, fluid reservoir 512, fluid pressurization and dispensing control system, and sensors 518 of system 500 can correspond, respectively, to electrode arrays 104, 304, 404, RF generator 110, fluid reservoir 112, and sensors 118 described above. However, the vapor therapy system 500 shown in FIG. 5, with the inclusion of a vapor delivery needle 514, can be better suited for accessing and treating small superficial veins and/or surface varicosities than the system 100 of FIG. 1.

Body 502 can be a rigid or semi-rigid elongate body, and can house electrode array 504. The delivery needle 514 can be disposed on a distal end of the body and be in fluid communication with the electrode array and fluid reservoir 512. In other embodiments, the delivery needle 514 can be disposed on a vapor delivery catheter, such as catheter 102 described above. In one embodiment the output from a vapor generation chamber is diverted away from the tip to maintain vapor quality while keeping the temperature of the tip low. Plunger 508 can be advanced to open a valve located at the distal end of a chamber and allow the developed vapor to flow from a vapor generation chamber 520 of the body through needle 514 into a patient. In another embodiment, Plunger 508 can be in fluid communication with a reservoir and an electrode can be in fluid communication with the reservoir, but the fluid is not sufficiently pressurized to cause significant flow. Plunger 508 can be advanced increasing the flow and pressure of the fluid which is heated by the electrode to generate vapor. A switch may be incorporated in Plunger 508 that is in electrical communication with the generator such that when Plunger 508 is advanced a signal is sent to the generator to initiate energy delivery to the electrode. In some embodiments, the plunger is replaced with a pump, such as the pumps described above. The delivery needle 514 is typically adapted to be inserted into a vein of a patient while the body 502 is positioned outside of a patient. However, in other embodiments, both the body and needle can be positioned inside the patient.

The needle can be disposed on a distal end of the delivery lumen of the elongate member described herein, for example. Once the vapor is generated and delivered out of the tip of the needle, it can easily travel through the vein and successfully traverse the tortuosities; catheter or needle access along the entire desired treatment length need not be achieved. Thus, a single, or a reduced number of needle sticks can be required to treat an extensive network of small or tortuous superficial veins. The needle can be a standard hypodermic needle or it can include other vapor ports. The outer shaft of the needle can be insulated, either passively by incorporating a low heat conducting material onto its outer surface or by including an active cooling insulating sleeve.

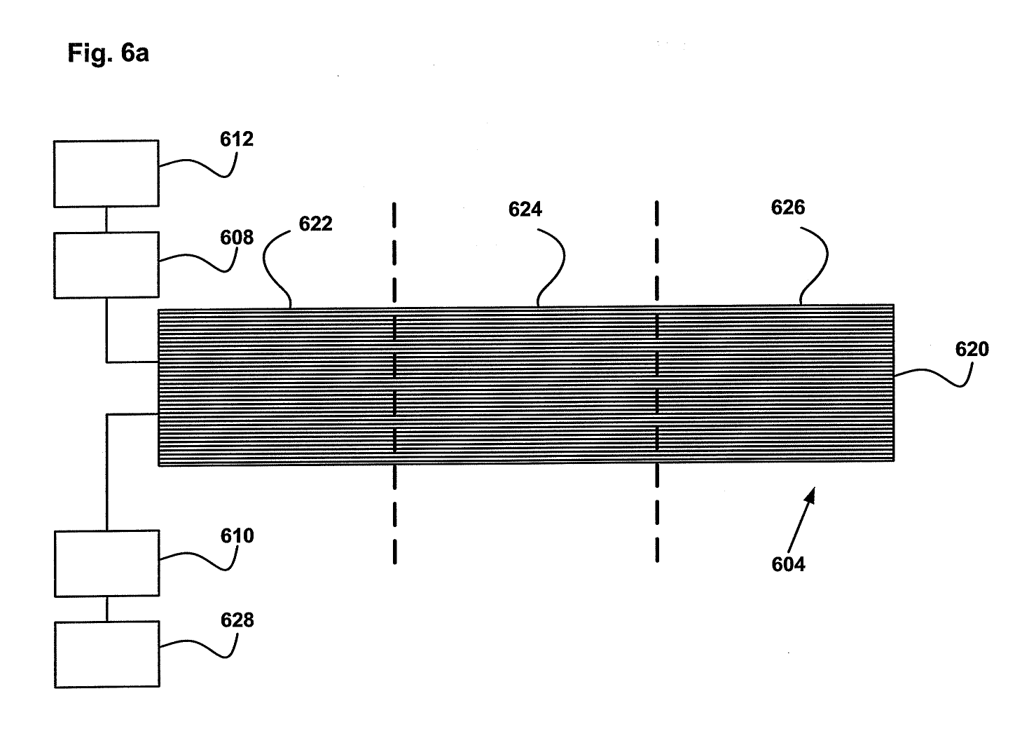

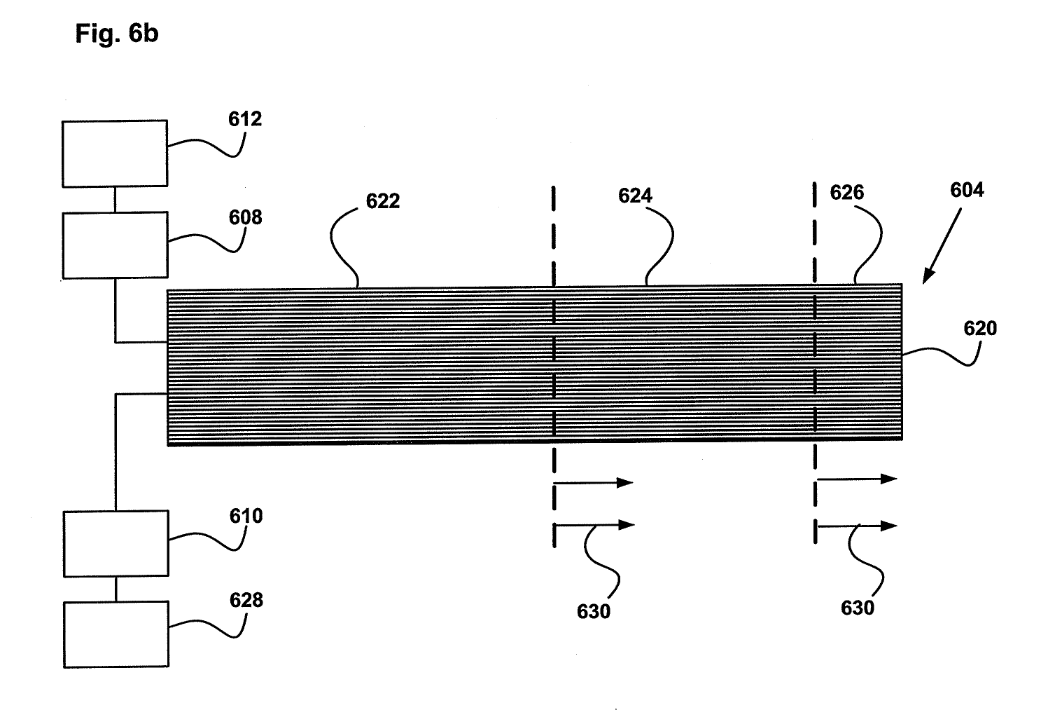

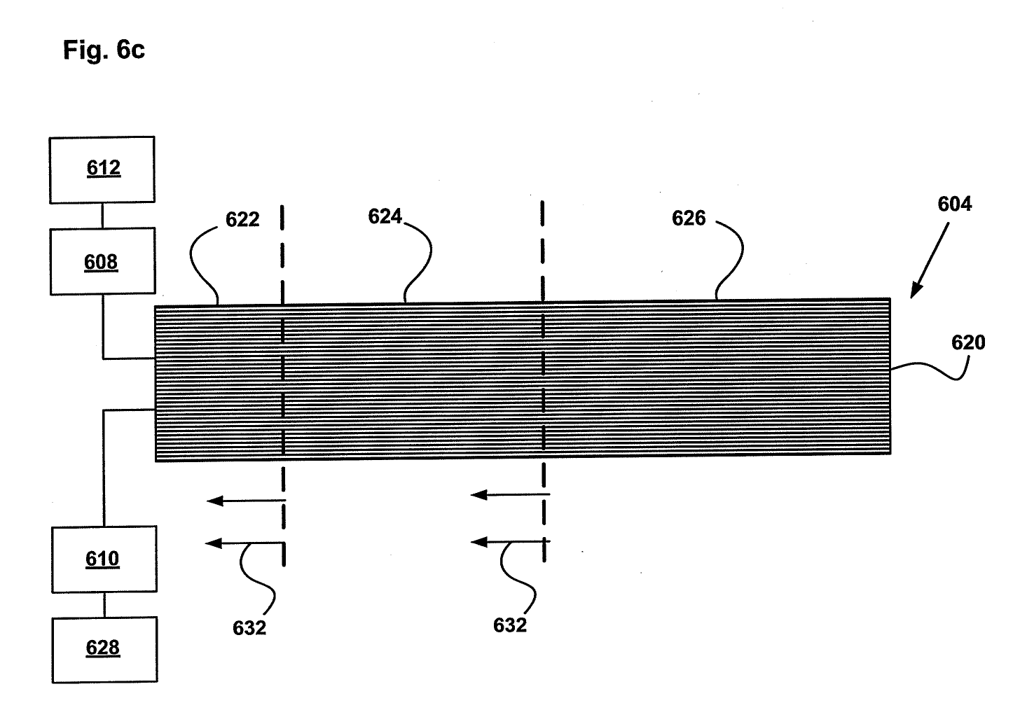

FIGS. 6a-6c illustrate a vapor generation chamber 620 adapted to generate a high temperature vapor or steam within an elongate member, such as the catheters or elongate bodies described above. In some embodiments, the vapor generation chamber can be disposed in an elongate body adapted to be inserted into a vein of a patient, such as for the vein lumen reduction therapy. In other embodiments, the chamber can be positioned outside of a patient. The vapor generation chamber 620 comprises an electrode array 604, which can be any of the electrode arrays described herein. Furthermore, the vapor generation chamber 620 can be connected to RF generator 610, pump 608, fluid reservoir 612, and controller 628, which can include of the RF generators and fluid reservoirs described herein or known in the art. As described above, the controller 628 can be integral to the RF generator in some embodiments and can also be separate from the RF generator.

A method for generating steam in a catheter or other elongate delivery device will now be discussed with reference to FIGS. 6a-6c. Referring to FIG. 6a, fluid can be delivered from fluid reservoir 612 to vapor generation chamber 620 by pump 608 or other pressurized means. The fluid can be a sterile saline or any other appropriate fluid. The fluid can be delivered at a constant or variable fluid flow rate. For example, in one embodiment a preferred constant flow rate is approximately 3 ml/minute. In some embodiments, controller 610 can control and adjust the fluid flow rate.

Next, the RF generator 610 can provide power to the electrodes 604 disposed in the vapor generation chamber 620. During generation of vapor, the fluid vapor generation chamber can be described with respect to three distinct regions, including warming region 622, steam generation region 624, and fully developed steam region 626. As the fluid flows through the vapor generation chamber and power is applied to the electrodes, the fluid is warmed in the warming region 622. When the fluid is heated to a sufficient temperature, such as 100 degrees Centigrade at atmospheric pressure, the fluid begins to transform into a vapor or steam in the steam generation region 624. All of the fluid can then be transformed into vapor by the time it reaches the fully developed steam region 626, after which it can exit the distal end of the vapor generation chamber and exit the catheter. If the pressure in the chamber is greater than atmospheric pressure, higher temperatures will be required and if it is lower than atmospheric pressure, lower temperatures will generate vapor.

During vapor generation, a signal corresponding to the vapor therapy system can be sensed to determine if a fluid has fully developed into a vapor before exiting a distal end of the vapor generation chamber. In some embodiments, the signal is sensed by the controller 628. Sensing whether the vapor is fully developed can be particularly useful for many surgical applications, such as in the treatment of varicose veins, where delivering high quality fully developed vapor to the veins results in more effective treatment. In one embodiment, the electrical impedance of the electrode array can be sensed. In other embodiments, the temperature of the fluid, temperature of the electrode array, fluid flow rate, pressure, or similar parameters can be sensed.

The sensed signal can indicate the respective sizes and/or positions of the warming region, steam generation region, and fully developed steam region within the vapor generation chamber. For example, as more fluid is contained within the vapor generation chamber 620, the impedance of the electrode array will decrease. If the power delivered to the electrode array remains constant as the impedance decreases, then the warming region, steam generation region, and fully developed steam region will shift distally in the vapor generation chamber, as indicated by arrows 630 in FIG. 6b. If the fully developed steam region 626 shifts too far in the distal direction, then fluid can exit the vapor generation chamber before fully transforming to a vapor. Alternatively, as less fluid is contained within the vapor generation chamber the impedance of the electrode array will increase. If the power delivered to the electrode array remains constant as the impedance increases, then the warming region, steam generation region, and fully developed steam region will shift proximally in the vapor generation chamber, as indicated by arrows 632 in FIG. 6c. As the fully developed steam region 626 shifts proximally, the higher quality vapor is produced, however the area must be optimized to ensure adequate vapor volume is produced.

Thus, the power delivered to the electrode array can be adjusted based on the sensed signal to fully develop vapor in the vapor generation region of the vapor generation chamber. This allows the vapor therapy system described herein to deliver a fully developed vapor from the catheter to a target tissue. In some embodiments, the power can be adjusted by controller 628. In other embodiments, the power can be adjusted by manipulation of the RF generator itself. If the sensed signal is the impedance of the electrode array, for example, then the power delivered to the electrode array can be increased as the impedance decreases to ensure that vapor is fully developed before leaving the vapor generation chamber. If the impedance increases, then the power delivered to the electrode array can be decreased to maintain the ideal amount of heating area within the vapor generation region.

In other embodiments other system parameters can be adjusted based on the sensed signal. In one embodiment, the flow rate of fluid into the vapor generation chamber can be adjusted to fully develop vapor in the vapor generation region of the vapor chamber. For example, if the sensed signal is the impedance of the electrode array, and the impedance increases, then the flow rate can be increased to maintain the relative position of the vapor generation region. Similarly, if the impedance decreases, the flow rate of fluid can be decreased. As described above, the system parameters can be adjusted automatically by controller 628, or alternatively, can be adjusted manually.

To provide heat therapy to the vein, the distal tip of the vapor generation catheter can be inserted into the patient's vein to place the distal tip at a desired location of vein lumen reduction. Ultrasound or fluoroscopy may be used to assist catheter placement.

Saline or other liquid can then provided by the pump to the vapor generation catheter at a rate of 1 to 5 ml/minute, for example, and the RF generator can provide power to the electrode array. The liquid flow rate and the level of RF power provided are interdependent, and one or both may be adjusted based on vein size, vein flow, and/or a sensed signal. In addition, a thermocouple or other temperature measurement device may be provided at the distal end of the vapor generation catheter to provide feedback to the generator based on vapor temperature. The generator may also be controlled based on electrode impedance, as described above, to ensure that only fully developed vapor is delivered from the catheter to the vein.

As vapor condenses on venous tissue, the released latent energy or heat of vaporization causes the vein walls to shrink inward. To treat a length of vein, the distal tip of the vapor generation catheter may be drawn back as vapor is delivered, such as at a rate of about 1 cm/sec. The actual speed of pull-back depends in large part on the diameter of the vein and the volume and temperature of the steam being delivered, of course.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A method for generating steam within a catheter, comprising:
delivering a fluid to a vapor generation chamber;
delivering power to an electrode array disposed on or in the vapor generation chamber;
sensing a signal of the electrode array; and
adjusting the power delivered to the electrode array based on the sensed signal to fully generate vapor in the vapor generation chamber.

2. The method of claim 1 wherein the signal is an impedance.

3. The method of claim 1 wherein the signal is a fluid flow rate.

4. The method of claim 1 wherein the signal is a steam temperature.

5. The method of claim 1 wherein the electrode array is a bipolar electrode array.

6. The method of claim 1 wherein the power delivered is decreased as the sensed impedance increases.

7. The method of claim 1 wherein the fluid is delivered at a constant flow rate.

8. The method of claim 1 wherein the fluid is delivered at a time varying rate.

9. The method of claim 1 wherein the fluid is delivered at a rate dependent upon the diameter of the vessel or organ being treated.

10. The method of claim 1 further comprising delivering vapor to a patient.

11. The method of claim 10 wherein the vapor is delivered to a vein of a patient.

12. The method of claim 11 wherein delivering the vapor to the vein reduces a lumen of the vein.

13. The method of claim 1 wherein the power is adjusted automatically by a controller.

14. The method of claim 1 wherein the power is adjusted manually.

15. The method of claim 1 wherein the fluid is saline.

16. The method of claim 1 where the fluid is electrically conductive.

17. A method of delivering therapy to a vein, comprising:
inserting a catheter into the vein;
delivering a fluid to a vapor generation chamber in the catheter;
delivering power to an electrode array disposed on the vapor generation chamber;
sensing a signal of the electrode array;
adjusting the power delivered to the electrode array based on the sensed impedance to fully generate vapor in the vapor generation chamber; and
delivering the fully generated vapor to the vein.

18. The method of claim 17 wherein the signal is an impedance.

19. The method of claim 17 wherein the signal is a fluid flow rate.

20. The method of claim 17 wherein the signal is temperature.

21. The method of claim 17 wherein the electrode array is a bipolar electrode array.

22. The method of claim 17 wherein the power delivered is decreased as the sensed impedance increases.

23. The method of claim 17 wherein the power delivered is increased as the sensed impedance decreases.

24. The method of claim 17 wherein the fluid is delivered at a constant flow rate.

25. The method of claim 17 wherein the fluid is delivered at a rate dependent upon the diameter of the vessel or organ being treated.

26. The method of claim 17 wherein the fluid is delivered at a rate dependent upon sensed impedance.

27. The method of claim 17 further comprising delivering vapor to a patient.

28. The method of claim 27 wherein the vapor is delivered to a vein of a patient.

29. The method of claim 28 wherein delivering the vapor to the vein reduces a lumen of the vein.

30. The method of claim 17 wherein the power is adjusted automatically by a controller.

31. The method of claim 17 wherein the power is adjusted manually.

32. A vapor generating device, comprising;
a vapor generation chamber, the vapor generation chamber having an electrode array disposed therein;
a fluid reservoir coupled to the vapor generation chamber;
an RF generator coupled to the electrode array; and
a controller configured to sense an impedance of the electrode array;
wherein the vapor generation chamber is adapted to transform a fluid from the fluid reservoir into a fully developed vapor within the device.

33. The device of claim 32 wherein the electrode array comprises a flattened configuration.

34. The device of claim 32 wherein the electrode array is disposed along an inner circumference of the catheter.

35. The device of claim 32 further comprising a second vapor generation chamber.

36. The device of claim 32 wherein the fluid is saline.

37. The device of claim 32 wherein the controller automatically adjusts a power delivered by the RF generator to the electrode array based on the sensed impedance.

38. The device of claim 32 further comprising a delivery needle coupled to the vapor generation chamber.
